(19) United States
(12) Patent Application Publication
(10) Pub. No.: US 2016/0175330 A1
(43) Pub. Date: Jun. 23, 2016

(54) METHOD OF TREATMENT USING AN ASSOCIATION OF LOSARTAN AND ACETYLSALICYLIC ACID, AND KIT

(71) Applicant: BIOLAB SANUS FARMACEUTICA LTDA, Taboão da Serra (BR)

(72) Inventor: Gilberto De Nucci, Campinas (BR)

(21) Appl. No.: 14/573,377

(22) Filed: Dec. 17, 2014

(51) Int. Cl. A61K 31/616; A61J 1/03; A61K 31/4178

(52) U.S. Cl. CPC A61K 31/616; A61K 31/4178; A61J 1/035

(57) ABSTRACT

The present invention relates to a method of treatment comprising administering an association of an antihypertensive agent and antiplatelet agent, wherein the antihypertensive agent is administered daily and the antiplatelet agent is administered every three days. The invention further comprises a kit comprising the association of the antihypertensive agent and the antiplatelet agent, packed so as to facilitate the compliance and the correct administration of the dosage of treatment by the patient.

### FIELD OF THE INVENTION

The present invention relates to a method of treatment comprising administering an association of an antihypertensive agent and antiplatelet agent, wherein the antihypertensive agent is administered daily and the antiplatelet agent is administered every three days. The invention further comprises a kit comprising the association of the antihypertensive agent and the antiplatelet agent, packed so as to facilitate the compliance and the correct administration of the dosage of treatment by the patient.

### BACKGROUND OF THE INVENTION

The joint administration of antihypertensive and antiplatelet agent is known in the art, and usually used to fight diseases such as heart failure, hypertension and ischemic heart disease.

Losartan is the first active ingredient of receptor type AT1 antagonists and nonpeptide angiotensin II antagonist, used as antihypertensive agent for the treatment of hypertension and heart failure. Losartan also reduces the combined risk of cardiovascular death, stroke and myocardial infarction in hypertensive patients with left ventricular hypertrophy, and offer renal protection in patients with type 2 diabetes and proteinuria.

In relation to the platelet antiplatelet agent, acetylsalicylic acid operates by the irreversible inhibition of the enzyme cyclooxygenase by means of an acetylation reaction of the serine residue 529, thus, keeping the catalytic site of this enzyme inaccessible to arachidonic acid. Thus, it inhibits prostaglandin H2 generation and, consequently, the generation of thromboxane A2. The non-release of thromboxane A2 in platelets inhibits platelet activation through the thromboxane receptor, and thus, an antiaggregant effect is produced. According to the prior art, the acetylsalicylic acid dosage for to prevent myocardial infarction, stroke and death in high-risk patients is 75 to 150 mg administered daily.

Therefore, based on the provisions of the art, one skilled in the art would be motivated to use the association of losartan and acetylsalicylic acid in the treatment of diseases such as heart failure, hypertension and ischemic heart diseases.

On the other hand, according to the disclosure in the prior art, it is widely known that the use of acetylsalicylic acid is associated with severe side effects, especially those related to gastrointestinal bleeding and gastric and intestinal ulceration. Due to the knowledge of these undesired side effects, studies have being conducted in order to achieve an effective treatment with absence or reduction of these effects.

Seeking to resolve the prior art problem, the researchers of this invention carried out studies using modifications in the administration frequency of the acetylsalicylic acid having as a goal the reduction of the side effects and the maintenance of the therapeutic efficacy. Among these studies, the researchers observed that when the acetylsalicylic acid was administrated every three days, the inhibitory effect of the platelet aggregation was sustained, whereas the PGE2 levels remained unaltered, which might be suggestive of a greater gastric protection, since the PGE2 is considered a gastric protector. Which differs from what was described in the prior art, where the daily use of the acetylsalicylic acid drastically reduces the PGE2 levels.

At last, the researchers developed a method of treatment comprising administering every three days the acetylsalicylic acid associated with the daily administration of losartan and a kit comprising the association of an antihypertensive agent and antiplatelet agent, packed so as to facilitate the compliance and the correct administration of the dosage of treatment by the patient.

### SUMMARY OF THE INVENTION

The present invention aims to provide a method of treatment comprising administering an association of losartan or its salt with acetylsalicylic acid, so as the losartan is administrated daily whereas the acetylsalicylic acid is administrated every three days.

Is also an object of this invention a kit comprising the association of an antihypertensive agent and antiplatelet agent, packed so as to facilitate the compliance and the correct administration of the dosage of treatment by the patient.

In a first embodiment, the invention relates to a method of treatment comprising the daily administration of losartan in unit dose of 25, 50 or 100 mg, associated with the administration every three days of acetylsalicylic acid in dose unit of 81 mg.

In a second embodiment, the invention relates to a kit in the form of blister pack to provide the proper sequential oral administration of the active ingredients of said association of losartan or salt thereof and acetylsalicylic acid, wherein said kit, according to FIG. 11, comprises:
(i) at least one unit dose of losartan or salt thereof, to be administered daily; and
(ii) at least one unit dose of acetylsalicylic acid to be administered every three days;

where the blister pack containing the unit doses mentioned in items (i) and (ii) in distinct dosage units is for use according to the frequency mentioned.

Preferably, the unit dose of losartan is 25, 50 or 100 mg, and a unit dose of acetylsalicylic acid is 81 mg. Being preferred that losartan is in the form of its potassium salt.

### BRIEF DESCRIPTION OF THE DRAWINGS

FIG. 1 shows the representative tracing for volunteers randomized to Treatment Arm A. The stimulation was performed with arachidonic acid 300 μg/ml. Where D0=the pretreatment curve; D15=the fifteenth day of the testing; D16=the sixteenth day of testing; D17=the seventeenth day of the testing.

FIG. 2 shows the representative tracing for volunteers randomized to Treatment Arm B. The stimulation was performed with arachidonic acid 300 μg/ml. Where D0=the pretreatment curve; D15=the fifteenth day of the testing; D16=the sixteenth day of testing; D17=the seventeenth day of the testing.

FIG. 3 shows the representative tracing for volunteers randomized to Treatment Arm A. Stimulation was done with adenosine diphosphate (ADP) 20 μM. Where D0=the pretreatment curve; D15=the fifteenth day of testing; D16=the sixteenth day of testing; D17=the seventeenth day of the testing.

FIG. 4 shows the representative tracing for volunteers randomized to Treatment Arm B. Stimulation was done with adenosine diphosphate (ADP) 20 μM. Where D0=pretreatment curve; D15=the fifteenth day of the testing; D16=the sixteenth day of testing; D17=the seventeenth day of the testing.

FIG. 5 shows the measurement values of thromboxane B2 (TXB2) in samples of plasma rich in platelets (PRP) stimulated with arachidonic acid in a concentration of 300 μg/mL on different days of testing for the Arm A volunteers.

FIG. 6 shows the measurement values of thromboxane B2 (TXB2) in samples of plasma rich in platelets (PRP) stimulated with arachidonic acid at a concentration of 300 μg/mL on different days of testing to Arm B volunteers.

FIG. 7 shows the measurement values of thromboxane B2 (TXB2) in samples of plasma rich in platelets (PRP) stimulated with adenosine diphosphate (ADP) at a concentration of 20 μM on different days of testing for Arm A volunteers. Where D0=the pretreatment curve; D15=the fifteenth day of the testing; D16=the sixteenth day of testing; D17=the seventeenth day of the testing.

FIG. 8 shows the measurement values of thromboxane B2 (TXB2) in samples of plasma rich in platelets (PRP) stimulated with adenosine diphosphate (ADP) at a concentration of 20 μM on different days of testing for Arm B volunteers. Where D0=the pretreatment curve; D15=the fifteenth day of testing; D16=the sixteenth day of testing; D17=the seventeenth day of testing.

FIG. 9 shows the bar graph representing the average of prostaglandin-E2 concentration in antrum biopsies (terminal portion of the stomach) collected during endoscopy, pre- and post-treatment, for volunteers randomized to Treatment Arm A.

FIG. 10 shows the bar graph representing the average of prostaglandin-E2 concentration in antrum biopsies (terminal portion of the stomach) collected during endoscopy, pre- and post-treatment, for volunteers in randomized Treatment Arm B.

FIG. 11 exemplifies the kit in the form of a blister pack comprising the active ingredients packed so as to meet the described dosage.

### DETAILED DESCRIPTION OF THE INVENTION

The present invention aims to provide a method of treatment comprising administering an association of losartan or its salt with acetylsalicylic acid, so as the losartan is administrated daily whereas the acetylsalicylic acid is administrated every three days. Is also an object of this invention a kit comprising the association of an antihypertensive agent and antiplatelet agent, packed so as to facilitate the compliance and the correct administration of the dosage of treatment by the patient.

The first embodiment of the present invention relates to a method of treatment, which comprises the daily administration of losartan in unit dose of 25, 50 or 100 mg, associated with the administration every three days of acetylsalicylic acid in dose unit of 81 mg, wherein said therapeutic method is capable of maintaining the desired therapeutic efficacy in the treatment of heart failure, hypertension and ischemic heart disease with reduction of the undesired side effects of acetylsalicylic acid.

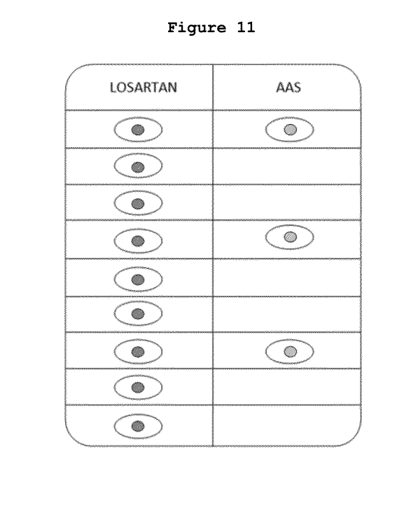

In a second embodiment, the present invention relates to a kit in the form of a blister pack, as shown in FIG. 11, that comprises:
(i) at least one unit dose of losartan or salt thereof, to be administered daily; and
(ii) at least one unit dose of acetylsalicylic acid to be administered every three days;

where the blister pack containing the unit doses mentioned in items (i) and (ii) in distinct dosage units, to provide the adequate sequential oral administration of the active ingredients of said association of losartan or its salt and acetylsalicylic acid.

In a more specific embodiment, the kit comprises the unit dose of 25, 50 or 100 mg of losartan potassium for daily administration, and the unit dose of 81 mg of acetylsalicylic acid for every three days administration.

The method of treatment, object of the present invention, was capable of significantly reducing the side effects associated with the administration of daily acetylsalicylic acid, without interfering with the bioavailability and the pharmaceutical efficacy of the acetylsalicylic acid, when administered to patients with heart failure, hypertension and ischemic heart disease.

It is known from the prior art that acetylsalicylic acid presents side effects to the gastrointestinal tract. On the other hand, the method of treatment, object of the present invention, provides therapeutic efficacy for the patients with heart failure, hypertension and ischemic heart disease, with reduction of the undesired side effects, especially lesions in the gastrointestinal tract, observed in the daily administration of acetylsalicylic acid.

### EXAMPLE I

### Study Methodology

To prove the administration of acetylsalicylic acid every three days, maintains its function of antiplatelet and presents reduction of side effects, tests were performed using two groups who were healthy, and who had no lesions in the gastrointestinal tract. One group (Arm A) received 30 days of daily doses of 50 mg of losartan potassium and 81 mg of acetylsalicylic acid. The second group (Arm B) received over the same 30 day period, daily doses of 50 mg of losartan potassium and doses of 81 mg of acetylsalicylic acid only every three days.

### EXAMPLE II

### Acetylsalicylic Acid Bioavailability

Control of bioavailability for acetylsalicylic acid in the analyzed individuals was performed through platelet aggregation tests, using arachidonic acid and adenosine diphosphate (ADP) as stimulants, and through the measuring of thromboxane by immunoenzymatic techniques.

Such control is based on tests to evaluate the competition between two compounds for binding to a specific antibody, namely, free thromboxane 2 (TXB 2) to be analyzed and present in the samples tested, and marked thromboxane 2 (TXB 2), attached to a molecule of acetylcholinesterase and with constant concentration (provided by the test kits).

The results of the bioavailability tests of acetylsalicylic acid which used arachidonic acid as a stimulant are shown in Figures (Arm A) and 2 (Arm B) of the present application.

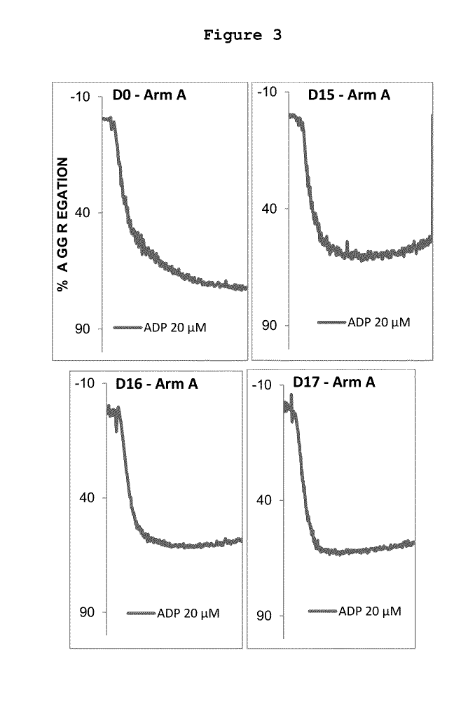

The test results when the adenosine diphosphate (ADP) was used as stimulant are shown in FIGS. 3 (Arm A) and 4 (Arm B).

The observation of the results leads to the conclusion that arms A and B showed almost the same level of platelet inhibition, demonstrating that the bioavailability of acetylsalicylic acid was not impacted by the higher frequency range of dosing.

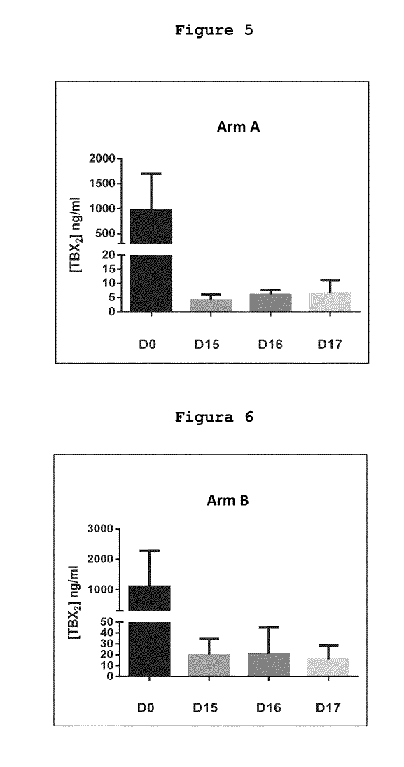

FIGS. 5, 6, 7 and 8 mentioned above support the conclusion by demonstrating the results of the measurement of thromboxane B2 (TXB2).

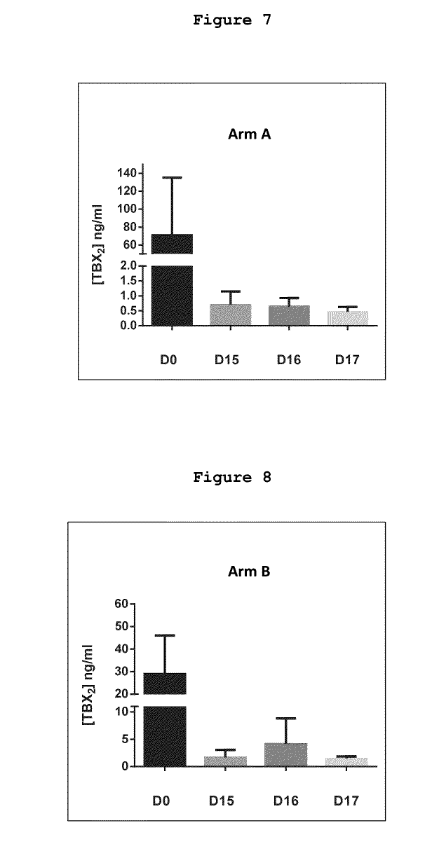

FIGS. 5 (Arm A) and 6 (Arm B) show the results of tests using arachidonic acid as a stimulant. While FIGS. 7 (Arm A) and 8 (Arm B) show the results of tests using adenosine diphosphate (ADP) as a stimulant.

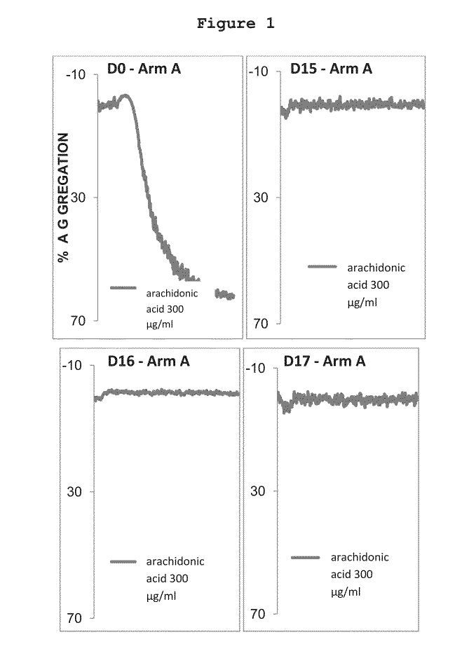

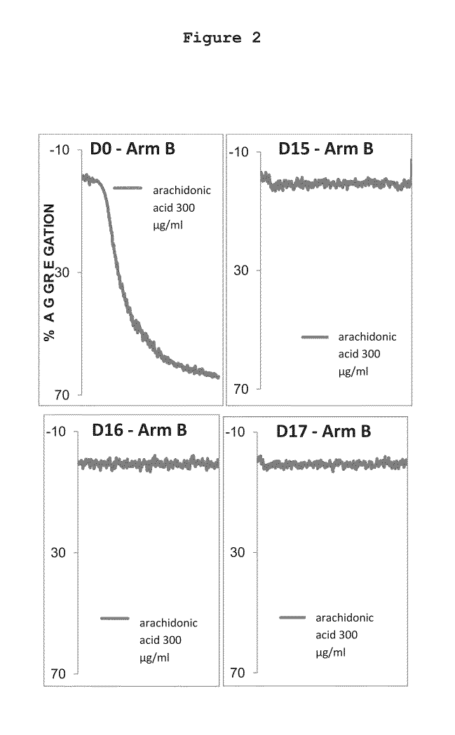

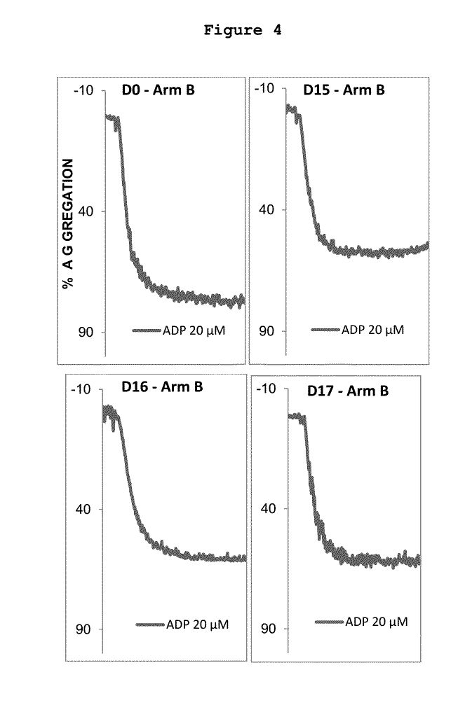

The test results shown in FIGS. 1 to 6 consider four periods: D0, which is the pre-test time D15—the fifteenth day of testing, D16—the sixteenth day of testing and D17—the seventeenth day testing. For Arm A, all samples were collected 24 hours after the last dose of acetylsalicylic acid, while in Arm B, in D15, the volunteers did not use the dose of acetylsalicylic acid for the previous 48 hours and, in D16 there were 72 hours without using acetylsalicylic acid according to the administration every three days and in D17 there were 24 hours without using acetylsalicylic acid.

Observation of the graphs of FIGS. 1 to 6 show that the differences between the results for Arm A and Arm B are negligible, and prove that there is satisfactory inhibition of the platelet cyclooxygenase enzyme in the two types of treatment—by using a daily dose of acetylsalicylic acid (Treatment Arm A) and using a dose of acetylsalicylic acid every three days (treatment arm B).

Based on the obtained results, the researchers proved the maintenance of the therapeutical efficacy of acetylsalicylic acid when administered every three days.

### EXAMPLE III

### Verification of the Reduction of Undesired Side Effects

To obtain this proof, endoscopic tests were performed on each volunteer, always before starting treatment and also after treatment, when biopsies were collected.

From these samples, collected from both Arm A and Arm B of the treatment, the inhibition of prostaglandin was measured.

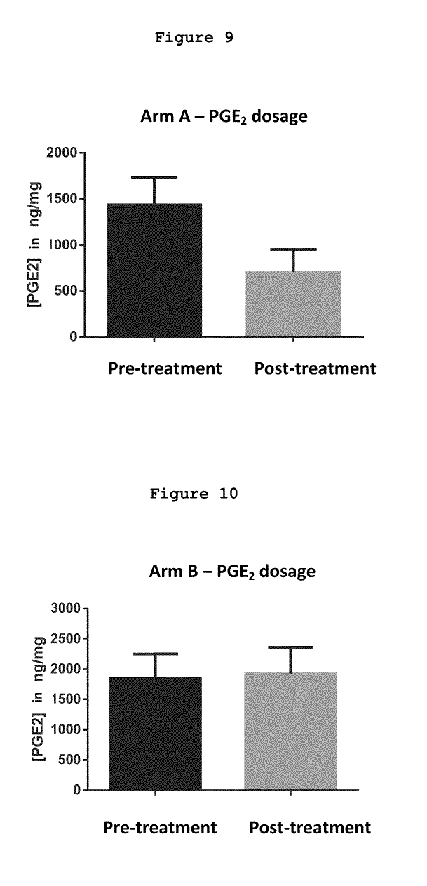

FIGS. 9 and 10 graphically show the difference observed in the inhibition of prostaglandin.

Analysis of the graphs shows emphatically that the use of acetylsalicylic acid, a substantial inhibitor of prostaglandin synthesis, and protective substance of the gastric mucosa administrated every three days did not alter the basal dose obtained in the pre-treatment, and that the use of acetylsalicylic acid on a daily basis (FIG. 9) caused a significant decrease in prostaglandin levels in post-treatment when compared with the pre-treatment basal dose.

With the aforementioned kit, patients who are affected by conditions such as heart failure, hypertension and ischemic heart disease, may maintain treatment through the administration of the combination with the reduction of adverse side effects, especially those related to the gastrointestinal tract, in an easier way.

FIG. 11 shows the blister pack embodiment of the present invention. The first vertical row (left side of the blister pack) contains the daily doses for the administrations of losartan, while the vertical row (right side of the blister pack) contains in the acetylsalicylic acid doses for administration every three days.

Although certain embodiments have been described, they are presented in an exemplary mode only, and are not intended to limit the scope of the invention. In fact, new embodiments described herein can be implemented in a variety of other forms, and furthermore; various omissions, substitutions and changes in the way in which the embodiments are described herein can be made without departing from the spirit of the invention. The claims which follow this description and its equivalents are considered to cover such forms or modifications insofar as they may be within the scope and spirit of the invention.

1. A method of treatment comprising administering an association of losartan or its salt with acetylsalicylic acid to treat diseases such as heart failure, hypertension and ischemic heart disease, wherein the losartan or salt thereof is administrated in a daily unit dose and the acetylsalicylic acid is administrated every three days.

2. The method according to claim 1, wherein the losartan salt is losartan potassium.

3. The method according to claim 1, wherein the dose of losartan potassium is selected from 25, 50 or 100 mg.

4. The method according to claim 1, wherein the dose of acetylsalicylic acid is 81 mg.

5. A kit in the form of a blister pack to provide the proper sequential oral administration of the active ingredients of an association of losartan or salt thereof and acetylsalicylic acid comprising:
(i) at least one unit dose of losartan or its salt for daily administration; and
(ii) at least one unit dose of acetylsalicylic acid for administration every three days;
where the blister pack contains the unit doses mentioned in items (i) and (ii) in distinct dosage units.

6. The kit according to claim 5, wherein the losartan salt is losartan potassium.

7. The kit according to claim 6, wherein the unit dose of losartan potassium is selected from 25, 50 ou 100 mg.

8. The kit according to claim 5, wherein the unit dose of acetylsalicylic acid is 81 mg.
